(19) United States
(12) Patent Application Publication
(10) Pub. No.: US 2013/0217748 A1
(43) Pub. Date: Aug. 22, 2013

(54) COSMETIC CLEANING AGENT

(71) Applicant: Henkel AG & Co, KGaA, (US)

(72) Inventors: Kirsten Sartingen, Brueggen (DE); Dirk Petersohn, Koeln (DE)

(73) Assignee: Henkel AG & Co. KGaA, Duesseldorf (DE)

(21) Appl. No.: 13/845,274

(22) Filed: Mar. 18, 2013

Related U.S. Application Data
(63) Continuation of application No. PCT/EP2011/073313, filed on Dec. 20, 2011.

(30) Foreign Application Priority Data
Dec. 20, 2010 (DE) 10 2010 063 578.2

(51) Int. Cl. A61K 8/88; A61Q 19/10

(52) U.S. Cl. CPC A61K 8/88; A61Q 19/10
USPC 514/423; 514/772.3; 514/561; 514/557; 514/565

(57) ABSTRACT

A cosmetic cleaning agent, comprises: in a cosmetic carrier: (a) greater than or equal to 12 wt % of a surfactant mixture comprising (i) at least one anionic surfactant and (ii) at least one zwitterionic/amphoteric surfactant in a weight ratio (i):(ii) of (2,5-3,5):1; (b) at least one cationic copolymer comprising (meth)acrylamide and dialkyldiallylammonium salts as the monomers; and (c) at least one opacifier and/or one pearlizer; wherein the quantities are based on the total weight of the cleaning agent. A method of producing a stable turbid and/or pearlescent cosmetic cleaning agent comprises: combining (a) at least one anionic surfactant and (b) at least one zwitterionic and/or an amphoteric surfactant to produce a surfactant combination; and combining, at room temperature, the surfactant combination with, (c) at least one cationic copolymer comprising (meth)acrylamide and dialkyldiallylammonium salts as the monomers, and (d) at least one opacifier and/or one pearlizer.

### TECHNICAL FIELD

The invention is in the field of cosmetics and relates to cleaning agents that comprise a particular mixture of surfactants, a specific cationic polymer and an opacifier or a pearlizer as well as methods for producing cosmetic cleaning agents and the use of cleaning agents to improve skin moisture.

### BACKGROUND

Cosmetic cleaning agents for the skin and/or the hair such as liquid soaps, shampoos, shower products, bath foams, as well as shower gels and washing gels have to possess, in addition to a good cleaning power, a good compatibility to the skin and/or the mucosa, and even with frequent use not lead to excessive degreasing or skin dryness.

Therefore modern skin cleaners often comprise additional care components such as specific polymers, so-called moisturizing components and/or emollients, in order to shift the moisture content of the skin as little as possible away from its natural equilibrium.

Incorporating and stabilizing such care components can be very time-consuming and costly, in particular when additional stabilizers and/or stabilization steps are needed.

Surfactant-containing cleaning agents for regulating skin moisture are disclosed in the application WO2006/076946 and comprise a mixture of yogurt proteins and at least one further active substance, selected from the group of plant extracts and/or plant milks and/or vitamins (derivatives).

A disadvantage with the shower products—disclosed principally in the examples of the abovementioned application—is that they can exhibit problems with stability.

In particular, when it was intended to dispense with the use of yogurt proteins for the production of shower products with a richer and creamier texture, the opacifier separated out.

It was found that the cationic polymer Polyquaternium-7 in the ahovementioned shower products can be replaced by cationic cellulose derivatives, such as for example Polyquatemium-10, without causing the opacifier to separate.

However, the incorporation and stabilization of the cationic cellulose derivatives in the shower products is disadvantageous because it requires a hot swelling process of the polymer in water as an additional production step.

There is still the need for stable cosmetic cleaning agents with a creamy texture and good foaming properties and which leave a pleasant skin feeling on the treated skin.

The intention of the present invention was to produce stable turbid or pearlescent cosmetic cleaning agents with very good care properties and skin compatibility. In particular, the intention was to produce cosmetic cleaning agents with a rich and creamy texture and which do not change the natural equilibrium of the protective sheath of the skin.

Furthermore, other desirable features and characteristics of the present invention will become apparent from the subsequent detailed description of the invention and the appended claims, taken in conjunction with the accompanying drawings and this background of the invention.

### BRIEF SUMMARY

In an embodiment, a cosmetic cleaning agent, comprises, in a cosmetic carrier: (a) greater than or equal to 12 wt % of a surfactant mixture comprising (i) at least one anionic surfactant and (ii) at least one zwitterionic/amphoteric surfactant in a weight ratio (i):(ii) of (2.5-3.5):1; (b) at least one cationic copolymer comprising (meth)acrylamide and dialkyldiallylammonium salts as the monomers; and (c) at least one opacifier and/or one pearlizer; wherein the quantities are based on the total weight of the cleaning agent.

In an embodiment, a method of producing a stable turbid and/or pearlescent cosmetic cleaning agent comprises: combining (a) at least one anionic surfactant and (b) at least one zwitterionic and/or an amphoteric surfactant to produce a surfactant combination; and combining, at room temperature, the surfactant combination with, (c) at least one cationic copolymer comprising (meth)acrylamide and dialkyldiallylammonium salts as the monomers, and (d) at least one opacifier and/or one pearlizer.

### DETAILED DESCRIPTION

The following detailed description of the invention is merely exemplary in nature and is not intended to limit the invention or the application and uses of the invention. Furthermore, there is no intention to be bound by any theory presented in the preceding background of the invention or the following detailed description of the Invention.

The cleaning agents, combined with water, should form a creamy, fine-cell foam that can be easily distributed on the skin, already imparting a perception of care during cleaning.

From the ecological point of view (and to save time), energetically intensive steps should be dispensed with as far as possible in the production of the agent.

The subject matter of the invention is a cosmetic cleaning agent that comprises in a cosmetic carrier

(a) greater than or equal to 12 weight percent (wt %) of a surfactant mixture comprising
(i) at least one anionic surfactant and
(ii) at least one zwitterionic/amphoteric surfactant in a weight ratio (i):(ii) of (2.5-3.5):1;

(b) at least one cationic copolymer that comprises (meth)acrylamide and dialkyldiallylammonium salts as the monomers; and

(c) at least one opacifier and/or one pearlizer;

wherein the quantities are based on the total weight of the cleaning agent.

The cleaning agents according to the invention comprise the components (a), (b) and (c) in a cosmetic carrier that can be preferably aqueous or aqueous-alcoholic.

The cosmetic carrier preferably comprises at least 40 wt % water.

Furthermore, the cosmetic carrier can comprise 0.01 to 50 wt %, preferably 0.05to 45 wt % and especially 0.1 to 40 wt % of at least one alcohol that can be selected from ethanol, ethyl diglycol, 1-propanol, 2-propanol, isopropanol, 1,2-propylene glycol, glycerin, 1-butanol, 2-butanol, 1,2-butane diol, 1,3-butane diol, 1-pentanol, 2-pentanol, 1,2-pentane diol, 1,5-pentane diol, 1-hexanol, 2-hexanol, 1,2-hexane diol, 1,6-hexane diol, sorbitol, benzyl alcohol, phenoxyethanol or mixtures of these alcohols.

The water-soluble alcohols are preferred.

Ethanol, ethyl diglycol, 1-propanol, 2-propanol, isopropanol, 1,2-propylene glycol, glycerin, benzyl alcohol and/or phenoxyethanol as well as mixtures of these alcohols are particularly preferred.

The cleaning agents according to the invention comprise, based on their total weight, at least 12 wt % of a surfactant mixture (a) that comprises

(i) at least one anionic surfactant and

(ii) at least one amphoteric surfactant in a weight ratio (i):(ii) of (2.5-3.5):1.

It was found that a stable dispersion of the pearlizer and/or opacifier (c) in a cleaning composition that comprises a cationic polymer (b) can be achieved only above a surfactant content (i)+(ii) of at least 12 wt %.

A stable dispersion of the pearlizer and/or opacifier (c) can also be achieved at higher surfactant contents (by still respecting the weight ratio (i):(ii)).

Excessively high surfactant contents should, however, be avoided on the grounds of skin protection; consequently it is inventively preferred that the cleaning agents comprise a maximum of 25 wt %, preferably a maximum of 20 wt % and in particular, a maximum of 15 wt % of the surfactant mixture (a).

Suitable anionic surfactants (i) that can be incorporated into the agents according to the invention include:

linear and branched fatty acids with 8 to 30 carbon atoms (soaps),

ether carboxylic acids of the Formula R—O—(CH₂—CH₂O)ₓ—CH₂—COOH, in which R is a linear or branched, saturated or unsaturated alkyl group with 8 to 30 carbon atoms and x=0 or 1 to 16,

acyl sarcosides with 8 to 24 carbon atoms in the acyl group,

acyl taurides with 8 to 24 carbon atoms in the acyl group,

acyl isethionates with 8 to 24 carbon atoms in the acyl group,

mono- and/or dialkyl esters of sulfosuccinic acid with 8 to 24 carbon atoms in the alkyl group and mono-alkyl polyoxyethyl esters of sulfosuccinic acid with 8 to 24 carbon atoms in the alkyl group and 1 to 6 oxyethyl groups,

alpha-olefin sulfonates containing 8 to 24 carbon atoms,

salts of alkyl sulfates and/or of alkyl polyglycol ether sulfates of the Formula R—O(CH₂—CH₂O)ₓ—OSO⁻X⁺, in which R is a preferably linear or branched, saturated or unsaturated alkyl group with 8 to 30 carbon atoms, x=0 or 1 to 12 and X is an alkali metal ion or an ammonium ion,

sulfonates of unsaturated fatty acids with 8 to 24 carbon atoms and 1 to 6 double bonds,

esters of tartaric acid and citric acid with alcohols,

the addition products of about 2-15 molecules of ethylene oxide and/or propylene oxide onto fatty alcohols with 8 to 22 carbon atoms,

alkyl and/or alkenyl ether phosphates of the Formula,

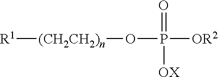

in which R¹ preferably stands for an aliphatic hydrocarbon group containing 8 to 30 carbon atoms, R² stands for hydrogen, a (CH₂CH₂O)ₙR¹ group or X, n for numbers between 0 and 10 and X for hydrogen, an alkali metal or alkaline earth metal or NR³R⁴R⁵R⁶, with R³ to R⁶, independently of each other, standing for a C₁ to C₄ hydrocarbon group,

Preferred anionic surfactants (i) are ether carboxylic acids of the abovementioned formula, acyl sarcosides with 8 to 24 carbon atoms in the acyl group, mono- and/or dialkyl esters of sulfosuccinic acid with 8 to 24 carbon atoms in the alkyl group and mono-alkyl polyoxyethyl esters of sulfosuccinic acid with 8 to 24 carbon atoms in the alkyl group and 1 to 6 oxyethyl groups, alpha-olefin sulfonates containing 8 to 24 carbon atoms and/or salts of alkyl sulfates and/or of alkyl polyglycol ether sulfates of the above mentioned formula.

Particularly preferred anionic surfactants (i) are straight chained or branched alkyl ether sulfates that comprise an alkyl group with 8 to 18 and especially 10 to 16 carbon atoms as well as 1 to 6 and especially 2 to 4 ethylene oxide units.

Further particularly preferred anionic surfactants (i) are straight chained or branched alkyl sulfonates that comprise an alkyl group with 8 to 18 and especially 10 to 16 carbon atoms.

The sodium, magnesium and/or triethanolamine salts of linear or branched lauryl, tridecyl and/or myristyl sulfates which have an ethoxylation degree of 2 to 4 are particularly preferred,

Suitable amphoteric/zwitterionic surfactants (ii) can be selected from compounds of the following Formulas (I) to (V), in which each R group stands for a straight chain or branched, saturated or a mono or polyunsaturated alkyl or alkenyl group with 8 to 24 carbon atoms,

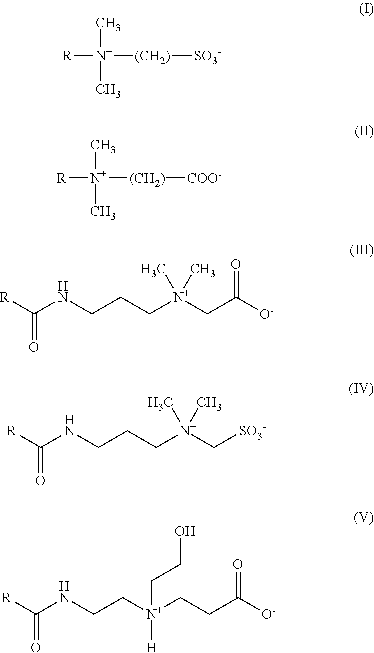

Particularly suitable amphoteric/zwitterionic surfactants (ii) are alkyl and/or alkylamidoalkyl betaines of the abovementioned Formulas (I) to (V).

A particularly suitable amphoteric/zwitterionic surfactant (ii) is the surfactant known by the INCI name Cocamidopropylbetain.

In a first preferred embodiment, a cosmetic cleaning agent according to the

invention preferably comprises

8.75 to 20 wt % of at least one anionic surfactant (i) from the group of the alkyl (ether) sulfates, the ether carboxylic acids, the mono and/or dialkyl esters of sulfosuccinic acid, the olefin sulfonates and/or the acyl sarcosides and

3.25 to 7.5 wt % of at least one surfactant (ii) known under the INCI name Cocamidopropylbetain,

wherein the weight ratio (i):(ii) is (2.5-3.5): 1 and
wherein the quantities are based on the total weight of the cosmetic cleaning agent.

Within this embodiment, it is more preferred if the weight ratio of the surfactants (i):(ii) is 2.55 to 3.25.

Within this embodiment, it is particularly preferred if the weight ratio of the surfactants (i):(ii) is 2,6 to 3.

Within this embodiment, it is especially preferred if the weight ratio of the surfactants (i):(ii) is 2,65 to 2.85.

The cleaning agents according to the invention comprise at least one cationic polymer (b) that comprises (meth)acrylamide and dialkyldiallylammonium salts as the monomers.

Preferred cationic polymers (b) comprise acrylamide and dialkyldiallylammonium salts such as for example halide and/or methosulfate salts.

Particularly preferred cationic polymers (b) comprise acrylamide and dialkyldiallylammonium chloride as the monomers and are known by the INCI name Polyquaternium-7.

Those particularly preferred cationic polymers known by the INCI name Polyquaternium-7 have an average molecular weight (weight average) of at least 500,000, preferably at least 1,000,000 and especially at least 2,000,000.

Suitable exemplary commercial products that can be incorporated into the cleaning agents according to the invention, and each comprising a 8-10% concentrated aqueous solution of a polymer known by the INCI name Polyquaternium-7, are:

Mirapol® 550 (available from Rhodia); Merquat® 550, Merquat® 550-L, Merquat® 2200 and Merquat® S (available from Ondeo Nalco); Polyquart® 701 , Dehyquart® 701 (available from Cognis); Conditioner® P7 (available from 3V Sigma), Galsilk® 700 (available from Galaxy Surfactants) and Tilamar® Quat 740 (available from DSM).

The cationic polymer(s) (b) is (are) incorporated into the cosmetic cleaning agents according to the invention—based on their total weight—preferably in an amount of 0.01 to 10 wt %, more preferably 0.05 to 7.5 wt %, particularly preferably 0.075 to 5 wt % and particularly 0.1 to 3 wt %.

The cleaning agents according to the invention comprise—based on their total weight—preferably 0,01 to 10 wt %, more preferably 0.05 to 7.5 wt %, particularly preferably 0.1 to 5 wt % and particularly 0.2 to 3 wt % of at least one opacifier and/or pearlizer (c).

Suitable opacifiers and/or pearlizers (c) can be selected from the group

of the mono and/or diesters of ethylene glycol, 1,2-propane diol and/or glycerin with C₈-C₂₄ fatty acids,

of the esters of polyethylene glycols with C₈-C₂₄ fatty acids and/or

of the styrene/acrylate copolymers.

Particularly suitable opacifiers and/or pearlizers (c) are known by the INCI names:

Glycol Distearate, such as for example the commercial product Cutina® AGS from Cognis, Glycol Monostearate, such as for example the commercial product Cutina® EGMS from Cognis, PEG-3 Distearate, such as for example the commercial product Genapol® TS from Clariant, PEG-2 Distearate, such as for example the commercial product Kessco® DEGMS from Akzo Nobel, Propylene Glycol Stearate, such as for example the commercial product Tegin® P from Goldschmidt and/or Styrene/Acrylates Copolymers such as for example the commercial products Joncryl® 67 from Johnson Polymers, Suprawal® WS from BASF and/or Acusol® OP 301 from Rohm & Haas.

Especially suitable opacifiers and/or pearlizers (c) for use in the cleaning agents according to the invention are known by the INCI names: Glycol Distearate, Glycol Monostearate, PEG-3 Distearate and/or Styrene/Acrylates Copolymer.

In a second preferred embodiment, a cosmetic cleaning agent according to the

invention preferably comprises

8.75 to 20 wt % of at least one anionic surfactant (i) from the group of the alkyl (ether) sulfates, the ether carboxylic acids, the mono and/or dialkyl esters of sulfosuccinic acid, the olefin sulfonates and/or the acyl sarcosides,

3.25 to 7.5 wt % of at least one surfactant (ii) known under the INCI name Cocamidopropylbetain,

wherein the weight ratio (i):(ii) is (2.5-3.5): 1,

0.075 to 5 wt % of at least one cationic polymer that comprises acrylamide and dialkyldiallylammonium salts such as halide and/or methosulfate salts as the monomers, and

0.1 to 5 wt % of at least one pearlizer and/or opacifier from the group of the compounds known by the INCI names Glycol Distearate, Glycol Monostearate, PEG-3 Distearate and/or Styrene/Acrylates Copolymer,
wherein the quantities are based on the total weight of the cosmetic cleaning agent,

Within this embodiment, cosmetic cleaning agents are more preferred which comprise

8.75 to 20 wt % of at least one anionic surfactant (i) from the group of the alkyl (ether) sulfates, the ether carboxylic acids, the mono and/or dialkyl esters of sulfosuccinic acid, the olefin sulfonates and/or the acyl sarcosides and

3.25 to 7.5 wt % of at least one surfactant (ii) known under the INCI name Cocamidopropylbetain,

wherein the weight ratio (i):(ii) is (2.5-3.5): 1,

0.1 to 3 wt % of at least one cationic polymer known by the INCI name Polyquaternium-7 and

0.2 to 3 wt % Styrene/Acrylates Copolymer,
wherein the quantities are based on the total weight of the cosmetic cleaning agent,

Within this embodiment, it is especially preferred if the weight ratio of the surfactants (i):(ii) is 2.55 to 3.25, more preferably 2.6 to 3 and especially 2.65 to 2.85.

To support the skin care and/or for a sustainable moistening of the skin, it is advantageous if the cosmetic cleaning agents according to the invention additionally comprise at least one active substance that positively influences the skin moisture and/or the natural acid-protective sheath of the skin in an amount of 0.001 to 10 wt %, preferably 0.005 to 7.5 wt %, particularly preferably 0.01 to 5 wt % and especially 0.02 to 4 wt %, wherein the quantities are based on the total weight of the cleaning agent.

Suitable exemplary active substances that positively influence the skin moisture and/or the natural acid-protective sheath of the skin, and can be incorporated into the cleaning agent without negatively influencing the desired texture, are

a. urea,

b. guanidine,

c. imidazole,

d. amino acids,

e. lactic acid or sodium lactate,

f. glycerin,

g. 1,2- and/or 1,3-diols,

h. taurine,

i. vitamins,

j. as well as the physiologically acceptable salts and/or derivatives of these active substances.

Suitable amino acids can be selected from the essential amino acids isoleucine, leucine, lysine, methionine, phenylalanine, threonine, tryptophan and/or valine, from the semi-essential amino acids arginine and/or histidine and/or from the non-essential amino acids alanine, aspartic acid, cysteine, cystine, glutamic acid, glycine, proline, serine and/or tyrosine.

Particularly suitable amino acids are glycine, L-arginine, L-proline and/or L-valine.

Suitable exemplary 1,2- and/or 1,3-diols are 1,2-propane diol, 1,2-pentane diol, 1,2-hexane diol, 1,2-dodecane diol and/or 1,3-propane diol. 1,2-Propane diol and/or 1,2-hexane diol are particularly suitable.

Suitable vitamins are understood to preferably include the following vitamins, provitamins and vitamin precursors as well as their derivatives:

Vitamin A: Retinol (vitamin A₁) as well as 3,4-didehydroretinol, (vitamin A₂) belong in the group of substances designated as vitamin A. β-carotene is the provitamin of retinol. Examples of suitable vitamin A components are vitamin A acid and its esters, vitamin A aldehyde and vitamin A alcohol as well as its esters such as the palmitate and acetate.

Vitamin B: The vitamin B group or the vitamin B complex include inter alia

Vitamin B₁ (thiamine)

Vitamin B₂ (riboflavin)

Vitamin B₃. The compounds nicotinic acid and nicotinamide (niacinamide) are often included under this designation.

Vitamin B₅ (pantothenic acid and panthenol). In the context of this group, panthenol is preferably used. Useable derivatives of panthenol are especially the esters and ethers of panthenol as well as catatonically derivatized panthenols. Specific representatives are for example, panthenol triacetate, panthenol monoethyl ether and its monoacetate as well as cationic panthenol derivatives.

Vitamin B₆ (Pyridoxine as well as Pyridoxamine and Pyridoxal).

Vitamin C (ascorbic acid): Its use in the form of the palmitate ester, the glucosides or phosphates can be preferred. The use in combination with tocopherols can also be preferred.

Vitamin E (tocopherols, especially α-tocopherol).

Vitamin F: The term “vitamin F” is usually taken to mean essential fatty acids, particularly linoleic acid, linolenic acid and arachidonic acid,

Vitamin H: The compound (3aS,4S,6aR)-2-oxohexahydrothieno[3,4-d]-imidazole-4-valeric acid denotes Vitamin H, for which the trivial name biotin has become accepted,

The cleaning agents according to the invention can preferably comprise vitamins, provitamins and vitamin precursors from the groups A, B, E and H.

Vitamins, provitamins and vitamin precursors of groups B and/or H such as nicotinamide, biotin, pantolactone and/or panthenol are particularly preferred,

In another preferred embodiment of the invention, the cosmetic cleaning agents preferably comprise at least two active substances from the group glycerin, lactic acid and/or sodium lactate, glycine, taurine, L-arginine, L-proline, L-valine, nicotinamide and/or panthenol.

In a third preferred embodiment, a cosmetic cleaning agent according to the invention preferably comprises

8.75 to 20 wt % of at least one anionic surfactant (i) from the group of the alkyl (ether) sulfates, the ether carboxylic acids, the mono and/or dialkyl esters of sulfosuccinic acid, the olefin sulfonates and/or the acyl sarcosides,

3.25 to 7.5 wt % of at least one surfactant (ii) known under the INCI name Cocamidopropylbetain,

wherein the weight ratio (i):(ii) is (2.5-3.5):1,

0.075 to 5 wt % of at least one cationic polymer that comprises acrylamide and dialkyldiallylammonium salts such as halide and/or methosulfate salts as the monomers,

0.1 to 5 wt % of at least one pearlizer and/or opacifier from the group of the compounds known by the INCI names Glycol Distearate, Glycol Monostearate, PEG-3 Distearate and/or Styrene/Acrylates Copolymer, and

0.005 to 7.5 wt % of at least one active substance that positively influences the skin moisture and/or the natural acid-protective sheath of the skin, selected from urea, guanidine, imidazole, amino acids, lactic acid or sodium lactate, glycerin, 1,2- or 1,3-diols, taurine, vitamins, and/or physiologically acceptable salts and/or derivatives of these active substances,
wherein the quantities are based on the total weight of the cosmetic cleaning agent,

Within this embodiment, cosmetic cleaning agents are more preferred which comprise

8.75 to 20 wt % of at least one anionic surfactant (i) from the group of the alkyl (ether) sulfates, the ether carboxylic acids, the mono and/or dialkyl esters of sulfosuccinic acid, the olefin sulfonates and/or the acyl sarcosides,

3.25 to 7.5 wt % of at least one surfactant (ii) known under the INCI name Cocamidopropylbetain,

wherein the weight ratio (i):(ii) is (2.5-3.5): 1,

0.01 to 3 wt % of at least one cationic polymer known by the INCI name Polyquaternium-7,

0.2 to 3 wt % Styrene/Acrylates Copolymer and

0.01 to 5 wt % of at least one active substance from the group glycerin, lactic acid and/or sodium lactate, glycine, taurine, L-arginine, L-proline, L-valine, nicotinamide and/or panthenol,
wherein the quantities are based on the total weight of the cosmetic cleaning agent.

Within this embodiment, it is especially preferred if the weight ratio of the surfactants (i): (ii) is 2.55 to 3.25, more preferably 2.6 to 3 and especially 2.65 to 2.85.

Within this embodiment, it is more preferred if the cleaning agents comprise at least two active substances from the group glycerin, lactic acid and/or sodium lactate, glycine, taurine, L-arginine, L-proline, L-valine, nicotinamide and/or panthenol.

Within this embodiment, it is particularly preferred if the cleaning agents comprise at least three active substances from the group glycerin, lactic acid and/or sodium lactate, glycine, taurine, L-arginine, L-proline, L-valine, nicotinamide and/or panthenol.

Within this embodiment, it is especially preferred if the cleaning agents comprise at least four active substances from the group glycerin, lactic acid and/or sodium lactate, glycine, taurine, L-arginine, L-proline, L-valine, nicotinamide and/or panthenol.

Besides the abovementioned active substances, the cleaning agents according to the invention can comprise additional active substances that lend advantageous properties to the cleaning agent.

The preferred optional active substances that can be incorporated in the cleaning agents according to the invention include, for example:

oily, waxy and/or fatty components which can be incorporated into the cleaning agents (based on their total weight) preferably in an amount of 0.01-20 wt %, particularly preferably 0.05-15 wt % and particularly 0.1-10 wt %,

non-ionic surfactants and/or non-ionic emulsifiers which can be incorporated into the cleaning agents (based on their total weight) preferably in an amount of 0.1 to 15 wt %, preferably 0.5 to 12.5 wt % and particularly 1 to 10 wt %,

cationic surfactants which can be incorporated into the cleaning agents preferably in amounts of 0.1 to 10 wt %, based on the total agent. Quantities of 0.2 to 7.5 wt % and particularly 0.3 to 5 wt % are particularly preferred and/or

protein hydrolyzates which can be incorporated into the cleaning agents preferably in amounts of 0.01 to 10 wt %, based on the total agent. Quantities of 0.1 to 5 wt %, particularly 0.1 to 3 wt %, are particularly preferred.

Suitable oily and/or fatty components can preferably be selected from mineral, natural and synthetic oily components and/or fats.

Normally, triglycerides and mixtures of triglycerides are used as the natural (vegetal) oils. Preferred natural oils are coconut oil, (sweet) almond oil, walnut oil, pear kernel oil, avocado oil, tea tree oil, soybean oil, sesame oil, sunflower oil, camellia oil, evening primrose oil rice grain oil, palm kernel oil, mango kernel oil, cuckoo flower oil, safflower oil, macadamian nut oil, grape seed oil, amaranth seed oil, argan oil, bamboo oil, olive oil, wheat germ oil, pumpkin seed oil, abutilon seed oil, hazelnut oil, safflower oil, canola oil, sasanqua oil, jojoba oil, rambutan oil, cocoa butter and shea butter.

Mineral oils, paraffin oils and isoparaffin oils as well as synthetic hydrocarbons are preferably used as the mineral oils. A hydrocarbon that can be used is, for example, the commercially available product 1,3-di(2-ethylhexyl)cyclohexane (Cetiol® S).

Silicone compounds come into consideration as the synthetic oils.

Silicones bring about excellent conditioning characteristics to hair. In particular they provide a better compatibility to wet and dry hair and in many cases are beneficial to the feel and softness of the hair.

It is therefore worthwhile to employ silicones in cosmetic hair treatment agents. Suitable silicones can be selected among:

i. volatile or non-volatile, linear or cyclic, crosslinked or non-crosslinked polyalkylsiloxanes, polyarylsiloxanes, polyalkylarylsiloxanes,

ii. polysiloxanes, which comprise one or more organofunctional groups in their general structure, selected from:
a. substituted or unsubstituted animated groups;
b. (per)fluorinated groups;
c. thiol groups;
d. carboxylate groups;
e. hydroxylated groups;
f. alkoxylated groups;
g. acyloxyalkyl groups;
h. amphoteric groups;
i. bisulfite groups;
j. hydroxyacylamino groups;
k. carboxy groups;
l. sulfonic acid groups; and
m. sulfate or thiosulfate groups;

iii. linear polysiloxane(A)-polyoxyalkylene(B) block copolymers of the type (A-B)ₙ with n>3;

iv. grafted silicone polymers having a non-silicone-containing organic backbone which consist of an organic main chain that is formed .from organic monomers that do not comprise silicone, onto which at least one polysiloxane macromer has been grafted in the chain as well as optionally on at least one chain end;

v. grafted silicone polymers having a polysiloxane backbone, onto which the non-silicone-containing organic monomers were grafted, which polymers possess a polysiloxane main chain, onto which at least one organic macromer that comprises no silicone has been grafted in the chain as well as optionally on at least one chain end;

vi. or their mixtures.
In addition, a dialkyl ether can serve as the oily component,

Dialkyl ethers suitable for use are particularly di-n-alkyl ethers containing a total of 12 to 36 carbon atoms and especially 12 to 24 carbon atoms such as, for example, di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether and di-tert.-butyl ether, diisopentyl ether, di-3-ethyldecyl ether, tert.-butyl n-octyl ether, isopentyl n-octyl ether and 2-methylpentyl n-octyl ether.

Di-n-octyl ether is particularly preferred and is commercially available under the name Cetiol® OE.

Fats are understood to mean fatty acids, fatty alcohols as well as natural and synthetic waxes that can be both in solid form as well as liquid in an aqueous dispersion.

Linear and/or branched, saturated and/or unsaturated fatty acids having 6-30 carbon atoms can be used as the fatty acids. Fatty acids containing 10-22 carbon atoms are preferred. Among these may be cited the isostearic acids, such as the commercial products Emersol® 871 and Emersol® 875, and isopalmitic acids such as the commercial product Edenor® IP 95, as well as all other fatty acids commercialized under the trade names Edenor® (Cognis). Further typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidonic acid, gadoleic acid, behenic acid and eurcic acid as well as their industrial mixtures.

Usually, the fatty acid fractions obtainable from coconut oil and palm oil are particularly preferred; in general, the addition of stearic acid is particularly preferred.

Saturated, mono or polyunsaturated, branched or linear fatty alcohols containing C₆ to C₃₀, preferably C₁₀ to C₂₂ and quite particularly preferably C₁₂ to C₂₂ carbon atoms can be added as the fatty alcohols. Decanol, octanol, octenol, dodecenol, decenol, octadienol, dodecadienol, decadienol, oleyl alcohol, erucyl alcohol, ricinol alcohol, stearyl alcohol, isostearyl alcohol, cetyl alcohol, lauryl alcohol, myristyl alcohol, arachidyl alcohol, capryl alcohol, caprin alcohol, linoleyl alcohol, linolenyl alcohol and behenyl alcohol, as well as the Guerbet alcohols, e.g. 2-ethylhexanol can be employed, this listing being intended as exemplary and not limiting. However, the fatty alcohols are preferably derived from naturally occurring fatty acids, usually obtained by reducing the fatty acid esters. Likewise, according to the invention, those tatty alcohols can be added that are obtained by reducing naturally occurring triglycerides like beef tallow, palm oil, peanut oil, oil of rapeseed, cotton seed oil, soya oil, sunflower oil and linen oil, or the fatty acid esters produced from their transesterification products with appropriate alcohols, thereby producing a mixture of different fatty alcohols. Such substances can be bought, for example, under the trade names Stenol®, e.g. Stenol® 1618 or Lanette®, e.g. Lanette® O or Lorol®, e.g. Lorol® C8, Lorol® C14, Lorol® C18, Lorol® C8-18, HD-Ocenol®, Crodacol®, e.g. Crodacol® CS, Novol®, Eutanol® G, Guerbitol® 16, Guerbitol® 18, Guerbitol® 20, Lsofol® 12, Isofol® 16, Isofol® 24, Isofol® 36, Isocarb® 12, Isocarb® 16 or Isocarb® 24. Of course, wool wax alcohols such as those that are commercially available for example, under the trade names Corona®, White Swan®, Coronet® or Fluilan® can also be inventively added.

Solid paraffins or isoparaffins, carnuba wax, beeswax, candelilla wax, ozocerite, ceresine, sperm wax, sunflower wax, fruit waxes such as for example apple wax or citrus wax, micro waxes of polyethylene (PE) or polypropylene (PP) can be added as the natural or synthetic waxes. These types of waxes are available, for example, from Kahl & Co., Trittau.

Further fats are for example

Ester oils. Ester oils are understood to mean the esters of C₆-C₃₀ fatty acids with C₂-C₃₀ fatty alcohols. The monoesters of fatty acids with alcohols containing 2 to 24 carbon atoms are preferred. Examples of the fatty acids moieties in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidonic acid, gadoleic acid, behenic acid and erucic acid as well as their industrial mixtures.

Examples of the fatty alcohol moieties in the ester oils are isopropyl alcohol, capronyl alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linoleyl alcohol, linolenyl alcohol, elaeostearyl alcohol, arachyl alcohol, gadoleyl alcohol, behenyl alcohol, erucyl alcohol and brassidyl alcohol as well as their industrial mixtures. Isopropyl myristate (Rilanit® IPM), isononanoic acid C₁₆₋₁₈ alkyl ester (Cetiol® SN), 2-ethylhexyl palmitate (Cegesoft® 24), stearic acid 2-ethylhexyl ester (Cetiol® 868), cetyl oleate, glycerin tricaprylate, coco fatty alcohol caprinate/-caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are particularly preferred.

Dicarboxylic acid esters such as di-n-butyl adipate, di-(2-ethylhexyl) adipate, di-(2-ethylhexyl) succinate and di-isotridecyl acetate as well as diol esters such as ethylene glycol dioleate, ethylene glycol di-isotridecanoate, propylene glycol di(2-ethylhexanoate), propylene glycol di-isostearate, propylene glycol di-pelargonate, butane diol di-isostearate, neopntyl glycol dicaprylate,

symmetrical, unsymmetrical or cyclic esters of carbon dioxide with fatty alcohols, e.g. glycerin carbonate or dicaprylyl carbonate (Cetiol® CC),

ethoxylated or non-ethoxylated mono, di and trifatty acid esters of saturated and/or unsaturated linear and/or branched fatty acids with glycerin, such as e.g. Monomuls® 90-018, Monomuls® 90-L12, Cetiol® HE or Cutina® MD.

Suitable exemplary non-ionic surfactants/emulsifiers include

C₈-C₃₀ fatty acid mono and diesters of addition products of 1 to 30 moles ethylene oxide to glycerin,

amine oxides,

addition products of 2 to 50 moles ethylene oxide and/or 0 to 5 moles propylene oxide, linear and branched fatty alcohols containing 8 to 30 carbon atoms, fatty acids containing 8 to 30 carbon atoms and alkyl phenols containing 8 to 15 carbon atoms in the alkyl group,

sorbitol esters of fatty acids and addition products of ethylene oxide to sorbitol esters of fatty acids such as, e.g., polysorbates,

sugar esters of fatty acids and addition products of ethylene oxide to sugar esters of fatty acids,

addition products of ethylene oxide to fatty acid alkanoiamides and fatty amines and/or alkyl polyglucosides.

For the case where a non-ionic surfactant is incorporated into the cleaning agents, then alkyl oligoglucosides, especially alkyl oligoglucosides, based on hydrogenated C_{12/14} coco alcohol with a degree of polymerization (DP) of 1-3 are preferred, e.g. those commercially available for example under the INCI name “Coco-Glucoside”.

Further preferred non-ionic surfactants are the C₈-C₃₀ fatty acid mono- and diesters of addition products of 1 to 30 moles ethylene oxide on glycerin; C₈-C₃₀ fatty acid mono and diesters of addition products of 1 to 10 moles ethylene oxide to glycerin are particularly preferred. That known by the INCI name PEG-7 Glyceryl Cocoate is particularly preferred.

Suitable exemplary cationic surfactants are quaternary ammonium compounds, esterquats and/or amido amines.

Preferred quaternary ammonium compounds are ammonium halides, particularly chlorides and bromides, such as alkyltrimethylammonium chlorides, dialkyidimethylammonium chlorides and trialkylmethylammonium chloride, e.g. ceryltrimethylammonium chloride, stearyltrimethylammonium chloride, distearyldimethylammonium chloride, lauryldimethylammonium chloride, lauryldimethyibenzylammonium chloride and tricetylmethylammonium chloride, as well as the imidazolium compounds known under the INCI designations Quaternium-27 and Quaternium-83. The alkyl chains of the abovementioned surfactants have preferably 10 to 18 carbon atoms.

Esterquats are known compounds, which comprise both at least one ester function and also at least one quaternary ammonium group as structural elements. Preferred esterquats are quaternized ester salts of fatty acids with triethanolamine, quaternized ester salts of fatty acids with diethanolalkylamines and quaternized ester salts of fatty acids with 1,2-dihydroxypropyldialkylamines. Such products are marketed, for example, under the trade names Stepantex®, Dehyquart®, Armocare® and Quartamin®.

The alkylamido amines are normally manufactured by the amidation of natural or synthetic fatty acids and fatty acid fractions with dialkylamino amines. A particularly suitable compound from this substance group is represented by stearamidopropyldimethylamine, commercially available under the designation Tegoamid® S 18.

Suitable protein hydro lyzates are product mixtures obtained by acid-, base- or enzyme-catalyzed degradation of proteins (albumins).

Protein hydrolyzates can be of both vegetal as well as of animal origin.

Animal protein hydrolyzates are, for example, protein hydrolyzates of elastin, collagen, keratin, silk and milk albumin, which can also be present in the form of their salts. Such products are marketed, for example, under the trade names Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Protein hydro lyzates of vegetal origin, e.g. soya, almond, rice, pea, potato and wheat protein hydro lyzates are preferred. Such products are available, for example, under the trade names Gluadin® (Cognis), DiaMin® (Diamalt), Lexein® (Inolex) and Crotein® (Croda).

Cationic protein hydro lyzates can also be used, wherein the basic protein hydrolyzate can originate from animals, for example from collagen, milk or keratin, from plants, for example from wheat, maize, rice, potatoes, soya or almonds, from marine life, for example from fish collagen or algae, or from biotechnologically obtained protein hydro lyzates. The cationic derivatives based on protein hydro lyzates can be obtained from the corresponding proteins by a chemical, particularly alkaline or acid hydrolysis, by an enzymatic hydrolysis and/or a combination of both types of hydrolysis. The hydrolysis of proteins generally produces a protein hydrolyzate with a molecular weight distribution from about 100 Daltons up to several thousand Daltons. Cationic protein hydro lyzates are preferred, whose base protein content has a molecular weight of 100 to 25,000 Daltons, preferably 250 to 5,000 Daltons. Moreover, cationic protein hydro lyzates are understood to include quaternized amino acids and their mixtures, Quaternization of the protein hydro lyzates or of the amino acids is often carried out using quaternary ammonium salts such as, for example, N,N-dimethyl-N-(n-alkyl)-N-(2-hydroxy-3-chloro-n-propyl) ammonium halides. Moreover, the cationic protein hydrolyzates can also be further derivatized. As typical examples of the cationic protein hydrolyzates and derivatives thereof may be mentioned the following commercially available products under their INCI names: Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Soy Protein, Cocodimonium Hydroxypropyl Hydrolyzed Wheat Protein, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Soy Protein, Quaternium-79 Hydrolyzed Wheat Protein, The cationic protein hydrolyzates and derivatives based on plants are quite particularly preferred.

Additional active substances, auxiliaries and additives that can be incorporated into the cosmetic cleaning agents according to the invention are for example:

UV-Filters,

Thickeners like gelatin or plant gums, for example agar-agar, guar gum, alginates, xanthane gum, gum arabica, karaya gum, locust bean flour, linseed gums, dextrans, cellulose derivatives, e.g. methyl cellulose, hydroxyalkyl cellulose and carboxymethyl cellulose, starch fractions and derivatives such as amylose, amylopectin and dextrins, clays and layered silicates such as e.g. bentonite or synthetic hydrocolloids such as e.g. polyvinyl alcohol, the Ca, Mg or Zn soaps,

structurants such as maleic acid and lactic acid,

dimethyl isosorbite,

cyclodextrins,

fiber structure improvers, particularly mono-, di- and oligosaccharides, such as, for example glucose, galactose, fructose, fruit sugar and lactose,

dye stuffs to color the composition,

substances for adjusting the pH, for example α- and β-hydroxycarboxylic acids such as citric acid, lactic acid, malic acid, glycolic acid,

active substances like bisabolol,

cholesterol,

chelating agents such as EDTA, NTA, β-alanine diacetic acid and phosphonic acids,

ceramides. Ceramides are understood to mean N-acylsphingosine (fatty acid amides of sphingosine) or synthetic analogs of such lipids (“pseudo-ceramides),

propellants such as propane-butane mixtures, N₂O, dimethyl ether, CO₂ and air,

antioxidants,

texturizers such as sugar esters, polyol esters or polyol alkyl ethers,

preservatives, such as for example sodium benzoate or salicylic acid,

viscosity regulators such as salts (NaCl).

The cleaning agents according to the invention preferably have a pH in a range that is gentle on the skin of 4 to 6.5, preferably 4 to 6 and especially 4.5 to 5.5.

The desired rich and creamy texture of the cosmetic cleaning agents according to the invention can be achieved particularly well in a certain viscosity range of the agent.

Accordingly, inventively preferred cosmetic cleaning agents preferably have a viscosity in the range 5,000 to 14,000 megaPascal seconds (mPa·s), preferably 6,000 to 13,000 mPa·s and especially 7,500 to 11,000 mPa·s (each measured with a Haake rotational viscosimeter VT550; 20 ° C.; measuring system MV; spindle MV II; 8 revolutions per minute (rpm)). Compositions of this viscosity can be conveniently and easily applied from a container onto the hand or the application surface, without running out through the fingers and dripping. At the same time the viscosity of the composition is low enough to ensure that it can be adequately dispersed on the application surface with the help of the hands.

The cleaning agents according to the invention exhibit outstanding properties when used on the skin.

They clean the skin in a particularly gentle manner, they are highly compatible and moisturize the skin during and after the cleaning. During and after cleaning, the skin feels smooth and silky.

The cleaning agents have a creamy texture and form a fine porous foam that can be easily dispersed on the skin.

The cleaning agents according to the invention also have the advantage that they are easily manufactured—preferably by simple mixing of the components at room temperature. A (hot) pre-swelling of the cationic polymer in water is not required.

A second subject matter of the invention is a method for producing stable turbid and/or pearlescent cosmetic cleaning agent, in which method in a first step a combination of surfactants of

(a) at least one anionic surfactant and

(b) at least one zwitterionic and/or an amphoteric surfactant is provided in a first step, and in a second step—preferably at room temperature—is combined with

(c) at least one cationic copolymer that comprises (meth)acrylamide and dialkyldiailylammonium salts as the monomers, and

(d) at least one opacifier and/or one pearlizer.

A third subject matter of the invention is the cosmetic use of an agent according to the invention for cleaning and caring for the skin as well as for improving the skin moisture.

### EXAMPLES

The following examples were produced (the quantities refer—in so far as otherwise stipulated—to wt %):

The viscosities of the shower products were adjusted to the desired viscosity of 5000 to 14,000 mPa·s, (each measured with a Haake rotational viscosimeter VT550; 20° C.; measuring system MV; spindle MV II; 8 rpm).

The following commercial products were employed in the shower products:

Shower products:

| | A | B | C | D | E |
|---|---|---|---|---|---|
| Sodium Laureth Sulfate | 13 | 13 | 13 | 14 | 15 |
| 70% active component | | | | | |
| Cocamidopropyl Betaine | 8.5 | 8.5 | 8.5 | 9.375 | 10 |
| 40% active component | | | | | |
| Plantacare ®¹ 818 | — | | 1 | 1 | |
| Conditioner ®² P7 | 1.5 | 1.5 | | | |
| Polyquart ®³ 701 | | | 1.5 | 1.5 | |
| Merquat ®⁴ 550 | | | | | 1.5 |
| Styrene-acrylic | 1 | 1 | 1 | — | |
| acid copolymer | | | | | |
| (40% in water) | | | | | |
| Glycol distearate | | | | 1.5 | |
| PEG-3 distearate | | | | | 2 |
| Panthenol 75% | 0.01 | 0.02 | 0.2 | 0.1 | 0.05 |
| Nicotinamide | 0.1 | 0.15 | | | |
| Glycerin 99.5% | 0.86 | 1 | 3 | 1 | — |
| Lactic acid 80% DAB | 0.1 | — | 0.15 | | 0.1 |
| Sodium lactate 60% | 0.1 | 0.1 | | | |
| Glycine | 0.003 | | | | |
| Taurine | | 0.006 | | | |
| L-Arginine | | | 0.008 | | |
| L-Proline | | | | 0.006 | |
| L-Valine | | | | | 0.009 |
| Gluadin ®⁵ WQ | — | 0.01 | 0.01 | — | |
| PEG-7 glyceryl cocoate | 0.6 | 0.8 | 0.5 | 0.3 | 0.8 |
| Sodium benzoate | 0.4 | 0.5 | 0.3 | 0.4 | 0.4 |
| Sodium salicylate | 0.23 | 0.25 | 0.2 | 0.23 | 0.23 |
| Perfume | q.s.* | q.s. | q.s. | q.s. | q.s. |
| Water | to100 | to100 | to100 | to100 | to100 |

* = quantum satis, e.g., as much as sufficient
¹C₈-C₁₄ alkyl polyglucoside (INCI: COCO-GLUCOSIDE); active component: 51-53%; Cognis
²Acrylamide dimethyldiallylammonium chloride copolymer (INCI: POLYQUATERNIUM-7); 3 V Sigma
³Acrylamide dimethyldiallylammonium chloride copolymer (INCI: POLYQUATERNIUM-7); Cognis,
⁴Acrylamide dimethyldiallylammonium chloride copolymer (INCI: POLYQUATERNIUM-7); Ondeo Nalco,
⁵INCI: LAURDIMONIUM HYDROXYPROPYL HYDROLYZED WHEAT PROTEIN; Cognis

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic cleaning agent comprising, in a cosmetic carrier:
(a) greater than or equal to 12 wt % of a surfactant mixture comprising
(i) at least one anionic surfactant and
(ii) at least one zwitterionic/amphoteric surfactant in a weight ratio (i):(ii) of (2.5-3.5): 1;
(b) at least one cationic copolymer comprising (meth)acrylamide and dialkyldiallylammonium salts as the monomers; and
(c) at least one opacifier and/or one pearlizer;
wherein the quantities are based on the total weight of the cleaning agent.

2. The cosmetic cleaning agent of claim 1, comprising
8.75 to 20 wt % of at least one anionic surfactant (i) comprising alkyl (ether) sulfates, ether carboxylic acids, mono and/or dialkyl esters of sulfosuccinic acid, olefin sulfonates and/or acyl sarcosides; and
3.25 to 7.5 wt % of at least one surfactant (ii) comprising Cocamidopropylbetain; wherein the weight ratio (i):(ii) is (2.5-3.5):1 and
wherein the quantities are based on the total weight of the cosmetic cleaning agent.

3. The cosmetic cleaning agent of claim 1, wherein the weight ratio (i):(ii) is 2.55 to 3.25.

4. The cosmetic cleaning agent of claim 3, wherein the weight ratio (i):(ii) is 2.6 to 3.

5. The cosmetic cleaning agent of claim 1, comprising 0.01 to 10 wt % of the cationic polymer (b), based on the total weight of the cleaning agent.

6. The cosmetic cleaning agent of claim 5, comprising 0.05 to 7.5 wt % of the cationic polymer (b), based on the total weight of the cleaning agent.

7. The cosmetic cleaning agent of claim 1, wherein the cationic polymer (b) comprises Polyquaterium-7.

8. The cosmetic cleaning agent of claim 1, comprising, 0.01 to 10 wt %, based on the total weight of the cleaning agent, of an opacifier and/or pearlizer (c) comprising
mono and/or diesters of ethylene glycol, 1,2-propane diol and/or glycerin with C₈-C₂₄ fatty acids,
esters of polyethylene glycols with C₈-C₂₄ fatty acids and/or
styrene/acrylate copolymers.

9. The cosmetic cleaning agent of claim 8, comprising 0.05 to 7.5 wt % of the opacifier and/or pearlizer (c).

10. The cosmetic cleaning agent of claim 1, comprising 0.001 to 10 wt %, based on the total weight of the cleaning agent, of at least one active principle that positively influences the skin moisture and/or the natural acid-protective sheath of the skin, selected from the group consisting of
a. urea,
b. guanidine,
c. imidazole,
d. amino acids,
e. lactic acid or sodium lactate,
f. glycerin,
g. 1,2- and/or 1,3-diols,
h. taurine,
i. vitamins, and
j. the physiologically acceptable salts and/or derivatives of these active principles.

11. The cosmetic cleaning agent of claim 10, comprising 0.005 to 7.5 wt % of the at least one active principle that positively influences the skin moisture and/or the natural acid-protective sheath of the skin.

12. The cosmetic cleaner of claim 10, comprising at least two active principles from the group glycerin, lactic acid and/or sodium lactate, glycine, taurine, L-arginine, L-proline, L-valine, nicotinamide and/or panthenol.

13. A method of producing a stable turbid and/or pearlescent cosmetic cleaning agent, comprising:
combining
(a) at least one anionic surfactant and
(b) at least one zwitterionic and/or an amphoteric surfactant to produce a surfactant combination; and
combining, at room temperature, the surfactant combination with,
(c) at least one cationic copolymer comprising (meth)acrylamide and dialkyldiallylammonium salts as the monomers, and
(d) at least one opacifier and/or one pearlizer.
